(19) United States
(12) Patent Application Publication
(10) Pub. No.: US 2016/0223556 A1
(43) Pub. Date: Aug. 4, 2016

(54) Chemiluminescent Protein Chip, Method and Kit for Detecting Seroglycoid Fucosylation Index

(71) Applicant: Beijing Youan Hospital, Capital Medical University, Beijing (CN)

(72) Inventors: Ning Li, Beijing (CN); Aiying Zhang, Beijing (CN); Shengqi Wang, Beijing (CN); Yang Ke, Beijing (CN); Yonghong Zhang, Beijing (CN)

(21) Appl. No.: 14/622,259

(22) Filed: Feb. 13, 2015

(30) Foreign Application Priority Data
Feb. 4, 2015 (CN) 201510058591.2

(51) Int. Cl. G01N 33/574; G01N 21/76; G01N 33/543

(52) U.S. Cl. CPC G01N 33/57488; G01N 33/54366; G01N 33/54353; G01N 21/76; G01N 2333/471; G01N 2440/38

(57) ABSTRACT

A chemiluminescent protein chip, kit and method for detecting seroglycoid fucosylation index, in the field of protein detection technology. The chemiluminescent protein chip includes a substrate slide, at least one detection subarea, detection spot areas and one control spot area. The detection spots are formed by fixed aplha fetoprotein (AFP)-specific antibodies.

### FIELD OF THE INVENTION

The invention relates to a protein detection technology, in particular to a chemiluminescent protein chip and method for detecting seroglycoid fucosylation index.

### BACKGROUND OF THE INVENTION

Alpha fetoprotein (AFP) produced by primary hepatic cancer has great difference from that generated by hepatitis, hepatic cirrhosis and other benign hepatic diseases in the carbohydrate chain. Compared with AFP generated by benign hepatic diseases, AFP generated by hepatic cancer has much higher fucosylation index. Fucose has the characteristic of binding to lens culinaris lectin. AFP can be categorized into AFP-L1, AFP-L2 and AFP-L3 according to their (fucose residues') different affinity for lens culinaris lectin, wherein AFP-L1 mainly comes from benign hepatic diseases, AFP-L2 mainly comes from pregnant women, and AFP-L3 is the fucosylation form of AFP and mainly comes from HCC. In 2005, FDA has formally approved to take AFP-L3 as one of markers of primary hepatic cancer. AFP-L3 has high specificity and sensitivity in early diagnosis, differential diagnosis, therapeutic effect evaluation and prognosis monitoring.

Fucose is a methylated hexose, exists in carbohydrate chains of various glycoproteins in tissues and serums and is called as protein-bound fucose (P-bf). AFP contains fucose residues in its carbohydrate chain, which heteroplasmon is called as fucosylated AFP (FucAFP). The percentage of the FucAFP in total AFP amount is called as fucosylation index (Fuol). The Fuol has important theoretical significance and clinical application significance and can be used as one important indicator in hepatic cancer diagnosis and prognosis application.

The conventional method of separating fucosylated proteins in serum comprises crossed affinity immunoelectrophoresis technique, affinity blotting, affinity chromatography, “dual-site sandwich” enzyme linked immunosorbent assay, LiBASys tester, μTASWako® i30 detection system technology and Hotgen Biotech glycosyl capture spin column pretreatment technology, wherein the phytolectin affinity immunoelectrophoresis technique and the μTASWako® i30 detection system technology require high conditions, complex operations and expensive reagents, which restricts their popularization and application; while the glycosyl capturing spin column increases the complexity of the operation because sample treatment and detection are performed separately.

### SUMMARY OF THE INVENTION

The invention provides a kit and detection method for quantitatively detecting AFP and/or FucAFP in a biological sample, based on the demand and blank of the prior art in quantitative detection technology of AFP and AFP-L3 in a serum, which are not only applicable to detecting AFP antigens in a serum, but also have generality in detecting other fucosylated proteins, and have the advantages of time saving, accuracy and convenience.

The invention adopts the following technical scheme:

In one aspect, the invention provides a chemiluminescent protein chip for detecting seroglycoid fucosylation index, characterized in that a substrate slide of the protein chip at least includes one detection subarea, and the one detection subarea is used for detecting one serum sample;

Two detection spot areas and one row control spot area are arranged in the detection subarea, wherein one of the detection spot areas contains detection spots formed from fixed alpha fetoprotein (AFP)-specific antibodies, the other detection spot area contains detection spots formed from fixed lens culinaris lectin, and the control spot area contains control spots formed from fixed bovine serum albumin (BSA);

Substances on all detection spots in the same detection spot area have the same concentration.

One detection spot area at least includes two detection spots.

The AFP specific antibodies are mouse anti-human AFP antibodies.

Several detection subareas are arranged on the substrate slide, each detection spot area includes 4 detection spots arranged in one row, and the control spot area includes 4 control spots arranged in one row; and the detection spots and the control spots are arranged in three parallel rows.

A bulge is arranged between the detection subareas as a physical partition.

In a further aspect, this invention provides a chemiluminescent kit for detecting seroglycoid fucosylation index, characterized in that it comprises any one of the chemiluminescent protein chips as mentioned above.

In one aspect, it also comprises an AFP standard substance, biotin-labeled AFP polyclonal antibodies, avidin horseradish peroxidase (HRP) and a HRP chemiluminescent substrate solution; the biotin-labeled AFP polyclonal antibodies are rabbit antibodies and come from a species different from that of the AFP specific antibodies fixed on the detection spots.

In one aspect, it also comprises conventional reagents Phosphate Buffered Saline (PBS) and PBS containing Tween® 20 (PBST) which are used for washing and diluting.

In a yet further aspect, this invention provides the use of any one of the aforementioned kit in detecting AFP and/or FucAFP and/or seroglycoid fucosylation index.

In still a further aspect, this invention provides a method for quantitatively detecting fucosylated protein, characterized in that it adopts any one of the chemiluminescent protein chips as mentioned above and comprises the following steps:

### (1) Sample Detection

Diluting a serum sample to be detected before drop-wise adding it on the detection subareas of the chemiluminescent protein chip, incubating, washing the detection subareas with PBST so as to remove nonspecific conjugates;

Adding the biotin-labeled AFP antibodies diluted with PBS, incubating, washing the detection subareas with PBST so as to remove nonspecific conjugates;

Adding the avidin HRP diluted with PBS, incubating, washing the detection subareas with PBST so as to remove nonspecific conjugates;

Adding the HRP chemiluminescent substrate solution, and scanning the protein chip with a chemiluminescent scanner to respectively obtain the chemiluminescence pixel values of AFP and fucosylated protein in the diluted serum sample to be detected;

### (2) Obtaining Standard Curve Equations of AFP and Fucosylated Protein:

X-coordinate of the standard curve equation of AFP is a gradient of concentration values of the AFP standard substance; Y-coordinate of the same is a series of chemiluminescence pixel values of AFP as detected in the step (1) by using the AFP standard substances with gradient concentrations as a series of samples to be detected;

X-coordinate of the standard curve equation of fucosylated protein is a gradient of concentration values of AFP-L3 in the AFP-L3 standard substance; Y-coordinate of the same is a series of chemiluminescence pixel values of fucosylated protein as detected in step (1) by using AFP-L3 standard substances with gradient concentrations as a series of samples to be detected; and the AFP-L3 standard substance is a serum containing fucosylated proteins (AFP);

(3) Plugging the chemiluminescence pixel value of AFP in the serum sample to be detected in step (1) into the standard curve equation of AFP to calculate out the AFP concentration of the diluted serum, and multiplying it with the dilution ratio to obtain the AFP concentration of the serum to be detected; plugging the chemiluminescence pixel value of fucosylated protein in the serum sample to be detected in step (1) into the standard curve equation of fucosylated protein to calculate out the fucosylated protein concentration of the diluted serum, and multiplying it with the dilution ratio to obtain fucosylated protein concentration of the serum to be detected;

The ratio of the fucosylated protein concentration of the serum to be detected to the AFP concentration of the serum to be detected is the fucosylation index.

The term “incubating” refers to incubating for 30 min at 37° C.

The invention provides a chemiluminescent protein chip for detecting seroglycoid fucosylation index. The chemiluminescent protein chip is based on the antibody-antigen-antibody sandwich reaction principle and the chemiluminiscence principle, and AFP-specific antibodies and lens culinaris lectin are also fixed on the chemiluminescent protein chip. The AFP-specific antibodies are used for binding all AFP (AFP-L1, AFP-L2 and AFP-L3) in a serum, and lens culinaris lectin is used for binding FucAFP. Control spots are also arranged. The total concentration of AFP and the concentration of FucAFP in the serum can be simultaneously detected under absolutely identical conditions, and the seroglycoid fucosylation index can be accurately obtained. The chemiluminescent protein chip provided by the invention at least includes one detection subarea which can detect one serum sample. In most embodiments, at least two detection subareas are preferably set, wherein one of the subareas is used for detecting a control serum, and the other subarea is used for detecting the serum sample to be detected. Further, in order to implement high throughput detection, multiple detection subareas are preferably set, such as three, four, five, six, seven, eight, nine or ten detection subareas, so that multiple serum samples can be detected on one chip, the clinical detection efficiency can be increased, and the cost can be reduced. As shown in FIG. 1, in one preferable embodiment of the invention, the one detection subarea includes 4 detection spots where AFP-specific antibodies are fixed, 4 detection sports where lens culinaris lectin is fixed and 4 control spots; the two kinds of detection spots and the control spots are arranged into three parallel rows.

The invention also provides a chemiluminescent kit for detecting seroglycoid fucosylation index, which includes the protein chip as mentioned above, conventional chemiluminescent reagents, standard curve equation data, etc.

The protein chip according to this invention has the following three advantages:

1. AFP and FucAFP in a serum are detected under substantially identical conditions to make sure the detected fucosylation index is more accurate and reliable.

2. Multiple samples can be simultaneously detected. Multiple duplicate samples or samples taken at different time points can be detected to obtain dynamic values, or various different samples can be detected. In a word, high throughput detection can be implemented. The detection cost is reduced and the detection efficiency is improved on the whole.

3. The amounts of serum samples and antibodies required for the protein chip described herein are greatly reduced: only 2.5 μl to 10 μl of original serum is needed, while 50 μl is needed for ELISA method detection; for antibody application to protein chin boards, 5 μl of antibody can be applied to at least 20 protein chips, used for detecting 200 serum samples; the antibody amount required is far lower than that of ELISA method, and the detection cost and expense are greatly reduced.

Meanwhile, the invention also provides a method for quantitatively detecting FucAFP using the kit, which comprises the following steps of: firstly, making serially diluted solutions of AFP with gradient concentrations to be detected by adopting a commercial AFP antigen standard substance, determining the chemiluminescence pixel value corresponding to each gradient concentration by using the chemiluminescence detection method, establishing a standard curve with the gradient concentrations as X-coordinate and the chemiluminescence pixel values as Y-coordinate and obtaining a linear regression equation.

The method for detecting seroglycoid fucosylation index provided herein comprises the steps of: on the protein chip as described above, by using the characteristics of specific binding between antibodies and antigens and specific binding between lens culinaris lectin and fucose, adding serum or plasma samples to incubate, then adding biotin-labeled AFP polyclonal antibodies and HRP-labeled avidin, finally adding HRP chemiluminescent substrate, and scanning and quantifying chemiluminescent signals by a chemiluminescent scanner; and plugging the acquired signal values into the pre-established linear regression equation to obtain the concentration of fucosylated proteins AFP-L3 in the sample.

The detection principle of the method described herein is somewhat different from common chemiluminescence immunoreaction. A compound “antibody—antigen—horse radish peroxidase labeled second antibody” is formed in the common chemiluminescence immunoreaction namely Elisa reaction, and the HRP chemiluminescent substrate solution is finally added to acquire the chemiluminescence value. However, because horse radish peroxidase itself contains fucose residues, if the second antibody is labeled by horse radish peroxidase, the fucose residues in the horse radish peroxidase will be bound to lens culinaris lectin so as to seriously interfere with detection values. Some experiments made herein prove that accurate fucosylation index cannot be obtained in this way, the false positive rate is very high, and very high fucosylation index can be obtained in normal serums. Hence, the chip and the method provided herein have the following principle of: orderly fixing AFP monoclonal antibodies and lens culinaris lectin on the protein chip, successively adding a serum to be detected, biotin-labeled AFP polyclonal antibodies and avidin HRP to respectively form “AFP McAb—AFP—biotin-labeled AFP PcAb—avidin HRP compound” and “lens culinaris lectin—AFP-L3—biotin-labeled AFP antibody—avidin HRP compound”, finally adding the HRP chemiluminescent substrate solution to incubate, scanning the protein chip by a chemiluminescent scanner to obtain chemiluminescence pixel values, and plugging the pixel values into the linear regression equation corresponding to the standard curve to obtain the concentrations of AFP and AFP-L3, and acquiring the percentage of FucAFP AFP-L3 in total AFP, namely fucosylation index.

Experimental results prove that the method described herein not only can be used for qualitative detection, but also can quantitatively detect AFP and FucAFP through chemiluminescence intensity. Compared with ELISA method, it has better sensitivity and specificity. In terms of time consuming, the ELISA method needs at least 3 hours, and the method disclosed herein only needs 1.5 hours. In terms of antibody usage amount, when the protein chips in the kit are used for antibody application, 5 μl of antibody can be applied to at least 20 chips to detect 200 serums, and the antibody amount required is far lower than that of the ELISA method. In terms of serum usage amount, the ELISA method needs 50 μl serum, while the kit and the detection method described herein only need 2.5 μl to 10 μl original serum to detect one serum sample. Hence, the kit and the detection method provided herein have the characteristics of high sensitivity, time saving, economy, etc., and the cost and time for serum protein detection can be greatly reduced.

In conclusion, the method disclosed herein combines the chemiluminiscence detection method, the standard curve and the protein chip technology and ensures high sensitivity, accuracy, high efficiency and low cost when the kit is used for AFP-L3 quantitative detection. The detection method provided herein is a feasible, reliable, economic, simple and time-saving method. The technical solution of the invention will provide an economic and reliable kit and detection method for detection FucAFP in a serum in a large-scale high-throughput way.

### BRIEF DESCRIPTION OF THE DRAWINGS

FIG. 1 shows the schematic of antibody application of AFP/lens culinaris lectin on the protein chip.

FIG. 2 shows the flow chart of protein chip in AFP/lens culinaris lectin antibody sandwich method.

FIG. 3 shows the results of AFP antigen detected by the AFP protein microarray;

AFP antibodies were applied to the slides with different concentrations, A: 1 mg/ml; B: 0.5 mg/ml; C: 0.25 mg/ml; Different concentrations of AFP antigen were used: 1. 80 ng/ml, 2. 40 ng/ml, 3. 20 ng/ml, 4. 10 ng/ml, 5. 5 ng/ml; Serum from HCC patients and serum from healthy persons: 6. HCC serum, 7. HCC serum, 8. blank control, 9. healthy serum, and 10. HCC serum.

FIG. 4 shows the standard curve chart and the regression equation of AFP detected by AFP protein microarray.

FIG. 5 is the scan chart of AFP antigen and serum samples detected by AFP protein microarray;

AFP antigen concentration: (1-5) 80 ng/ml, 40 ng/ml, 20 ng/ml, 10 ng/ml, 5 ng/ml; HCC serum 6-10. AFP antibodies concentration coated to the slides were 0.5 mg/ml.

FIG. 6 is the scan chart of AFP-L3 standard substances detected by AFP/lens culinaris lectin applied chips;

AFP antibodies and lens culinaris lectin were applied to the slides, A: AFP antibodie, 0.5 mg/ml; B: lens culinaris lectin 4 mg/ml; Different concentrations of AFP-L3 in serum samples were used: (1-5) 100 ng/ml; 50 ng/ml; 25 ng/ml; 12.5 ng/ml; 6.2 5 ng/ml; (6-9) 100 ng/ml; 50 ng/ml; 25 ng/ml; 12.5 ng/ml. (10) blank control.

FIG. 7 shows the standard curve chart and the regression equation of AFP-L3 detected by AFP/lens culinaris lectin protein microarray.

FIG. 8 shows the scan chart of hepatic cancer and normal serum samples detected by AFP/lens culinaris lectin applied chips;

8 chips are used for detecting 39 hepatic cancer serum samples, 32 normal healthy serum samples and 9 blank controls.

### DETAILED DESCRIPTION OF THE INVENTION

The following embodiments are intended to further describe the invention in detail, but do not restrict the scope of the invention. Unless otherwise specified, operations used in the following embodiments are conventional methods, and reagents adopted are commercially available.

### Main Instrument Equipment

Chemiluminiscent scanner, researched and developed by Academy of Military Medical Sciences.

### Main Reagents and Sources Thereof

Mouse monoclonal antibody for AFP (Shenzhen Feipeng Company), lens culinaris lectin (Sigma Company), aldehyde substrate chips (Shanghai Baiao Company), biotin-labeled rabbit antibodies (Abcam Company), avidin-HRP (Abcam Company), HRP chemiluminescent substrate solutions A and B, to be mixed according to the proportion of 1:1 and used immediately after preparation (Millipore Company).

### Example 1

### Preparation and Application of the Protein Chip

Reagents and instruments used in experiments: mouse-induced monoclonal antibody AFP (Shenzhen Feipeng Company), lens culinaris lectin (Sigma Company), aldehyde substrate chips (Shanghai Baiao Company), biotin-labeled rabbit-induced antibodies (Abcam Company), avidin-HRP (Abcam Company), and chemiluminiscent scanner (researched and developed by the laboratory of professor Wang Sheng-Qi from Academy of Military Medical Sciences).

NaCl 8 g, KCl 0.2 g, Na2HPO41.44 g and KH2PO4 0.24 g, pH 7.4, volume 1 L PBS formula:

PBS, 1L+Tween-20, 1 ml PBST formula:

Chips are aldehyde substrate chips (Shanghai Baiao Company); each chip includes 10 detection grids (detection subareas); each grid detects one serum; and 10 serums are detected at one time.

In each detection grid, mouse monoclonal antibody for AFP (Shenzhen Feipeng Company) and lens culinaris lectin (Sigma Company) are successively applied to the chips for four times, wherein the monoclonal antibody for AFP is applied at a concentration of 0.5 mg/ml, the lens culinaris lectin is applied at a concentration of 4 mg/ml, both of which are applied into two rows of eight detection spots; 10% bovine serum albumin (BSA) is used as negative control and is also applied for four times to form control spots.

Operation Procedure of the Protein Chip:

Detect tumor markers in dynamic serum samples of healthy control group and hepatic cancer experimental group by using the protein chip prepared as above.

Dropwise add 10 μl serum sample (or 2.5 μl after diluting 4 times) on chips, incubate for 30 min at 37° C., so that AFP in the serum can be specifically bound to corresponding (mouse) antibodies on the chips by using specific binding between antigens and antibodies and specific binding between lens culinaris lectin and focuse to form a compound of antigen and antibody (mouse), and lens culinaris lectin is bound to focuse to form a compound of lens culinaris lectin and antigen.

Wash the chips for 4 times with PBST to remove non-specific binding, add PBS-diluted biotin-labeled rabbit primary antibodies, and incubate for 30 min at 37° C. Rabbit antibodies are bound to antigens to form a compound of mouse antibody—AFP (focuse)—biotin-labeled rabbit antibody and a compound of lens culinaris lectin—(AFP) focuse—biotin-labeled rabbit antibody.

Wash the chips for 4 times with PBST to remove non-specific bindings, add PBS-diluted avidin HPR, and incubate for 30 min at 37° C. Biotin is bound to avidin to form “mouse antibody—AFP (focuse)—biotin-labeled rabbit antibody—avidin HRP compound” and “lens culinaris lectin—(AFP) focuse—biotin-labeled rabbit antibody-avidin HRP compound”.

Wash the chips for 4 times with PBST to remove non-specific bindings, add chemiluminiscent HRP substrate, incubate for 30 min at 37° C., and scan them with the chemiluminiscent scanner.

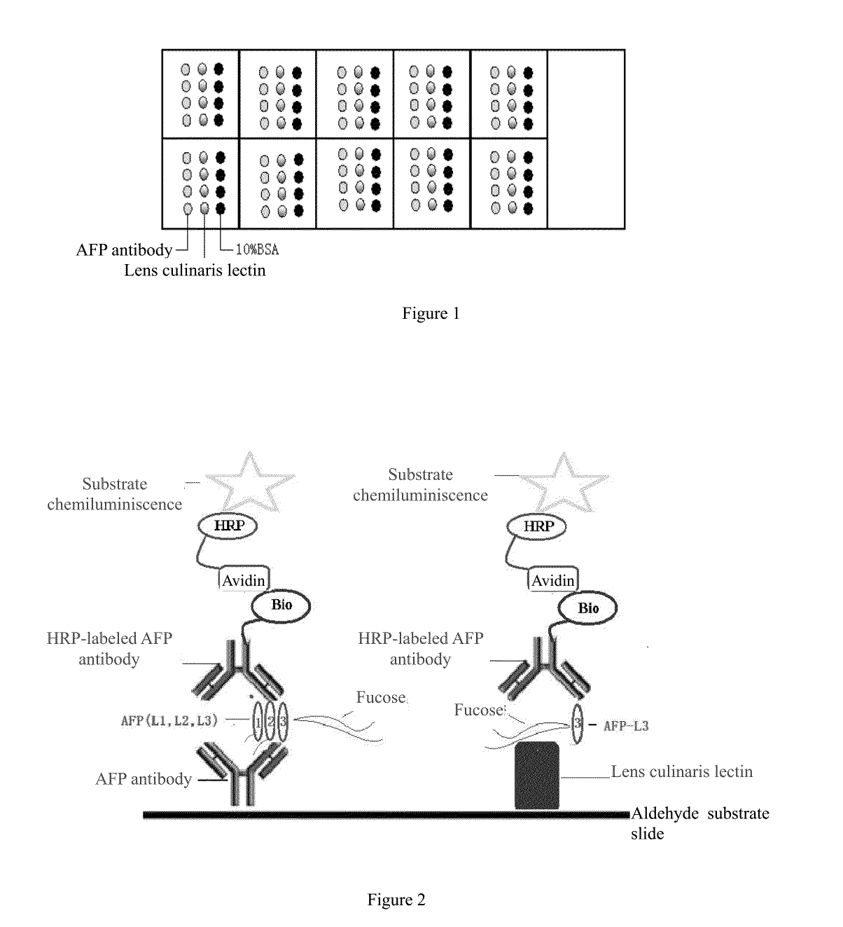

Chemiluminiscent pixel on a solid phase carrier is positively correlated to the amount of detected antigens in a sample, and the content of antigens to be detected can be determined by determining the pixel value in the compound. Chip application antibody (mouse primary antibody) and antibody for detection (rabbit-induced primary antibody) are respectively derived from different species of animals. FIG. 2 shows the flow chart of protein chip in the antibody sandwich method;

### Example 2

### Establishment of the Detection Method Described Herein

(1) Standard Curve and Regression Equation a.

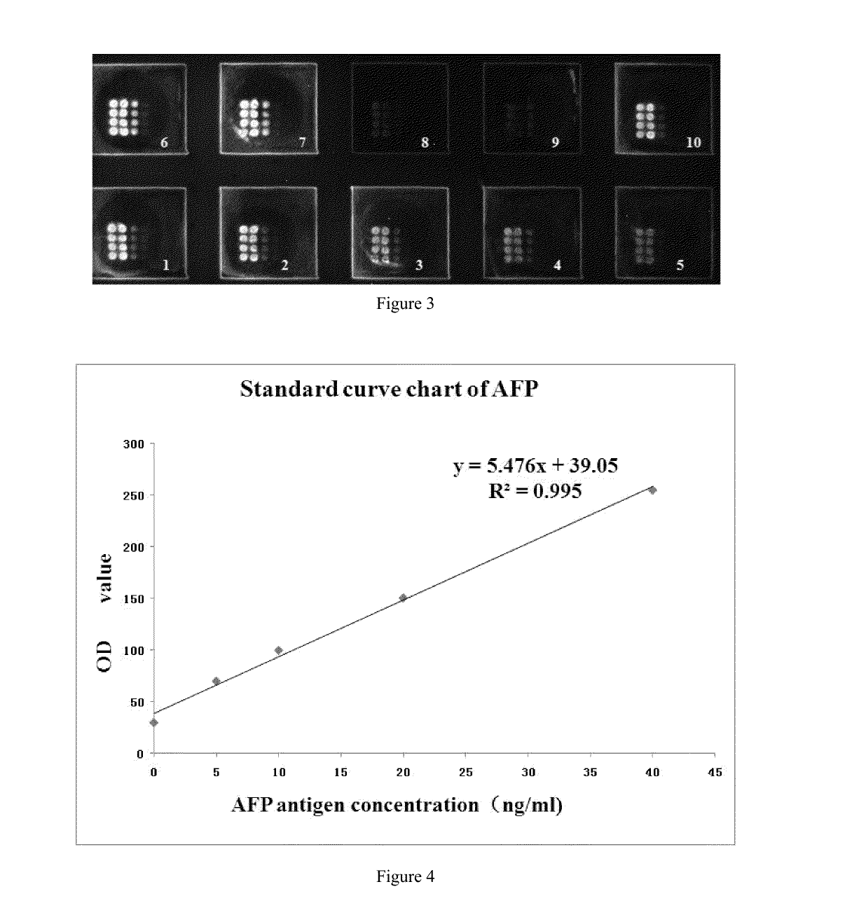

Commerical AFP antigens (Abcam Company) are set into different concentration gradients: (1-5) 80 ng/ml, 40 ng/ml, 20 ng/ml, 10 ng/ml, 5 ng/ml, 6. hepatic cancer serum, 7. hepatic cancer serum, 8. blank control, 9. healthy serum and 10. hepatic cancer serum (FIG. 3, antibodies applied in chip are AFP 2 mg/ml, 1 mg/ml, 0.5 mg/ml and 0.25 mg/ml).

The operation procedure and protein chips in the example 1 are used to detect each concentration gradient of the AFP standard substance, and the detection scan result is shown in FIG. 3. The detection results are used for drawing the standard curve chart, with the concentrations of the standard substance as X-coordinate and the pixel values as Y-coordinate, on a coordinate paper. Find out the corresponding concentration through the standard curve according to the pixel value of the sample; multiply it by the dilution ratio; or calculate out the linear regression equation of the standard curve using the concentration and OD value of the standard substance, plug the OD value of the sample into the equation to calculate out the concentration of the sample, and multiply it by the dilution ratio to obtain the actual concentration of the sample. The standard curve and the regression equation are shown in FIG. 4.

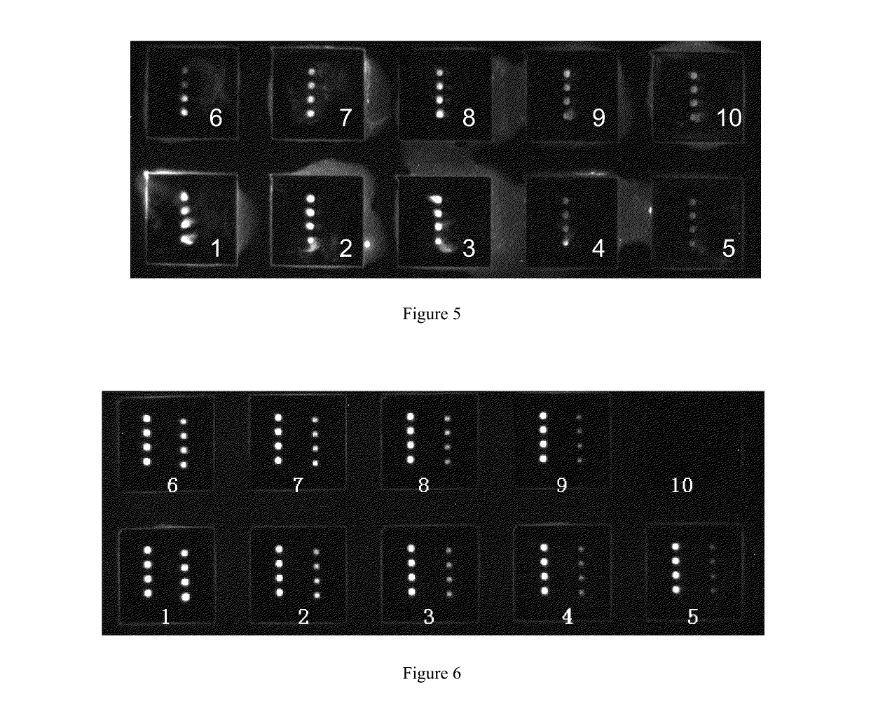

Commercial AFP antigens (Abcam Company) are set into different concentration gradients: (1-5) 80 ng/ml, 40 ng/ml, 20 ng/ml, 10 ng/ml, 5 ng/ml, 6. hepatic cancer serum, 7. hepatic cancer serum, 8. hepatic cancer serum, 9. hepatic cancer serum and 10. hepatic cancer serum (FIG. 5, antibodies applied in chip are AFP 0.5 mg/ml).

The operation procedure and protein chips in the example 1 are used to detect each concentration gradient of the AFP standard substance, and the detection scan result is shown in FIG. 3. The detection results are used for drawing the standard curve chart, with the concentration of the standard substance as X-coordinate and the pixel value as Y-coordinate, on a coordinate paper. Find out the corresponding concentration through the standard curve according to the pixel value of the sample; multiply it by the dilution ratio; or calculate out the linear regression equation of the standard curve using the concentration and OD value of the standard substance, plug the OD value of the sample into the equation to calculate out the concentration of the sample, and multiply it by the dilution ratio to obtain the actual concentration of the sample. The standard curve and the regression equation a are shown in FIG. 4.

(2) Standard Curve and Regression Equation b.

A serum with known AFP-L3 concentration is diluted in multiple proportions, and set into different concentration gradients: (1-5) 200 ng/ml, 100 ng/ml, 50 ng/ml, 25 ng/ml, 12.5 ng/ml, (6-9) 200 ng/ml, 100 ng/ml, 50 ng/ml, 25 ng/ml. 10. blank control (FIG. 6).

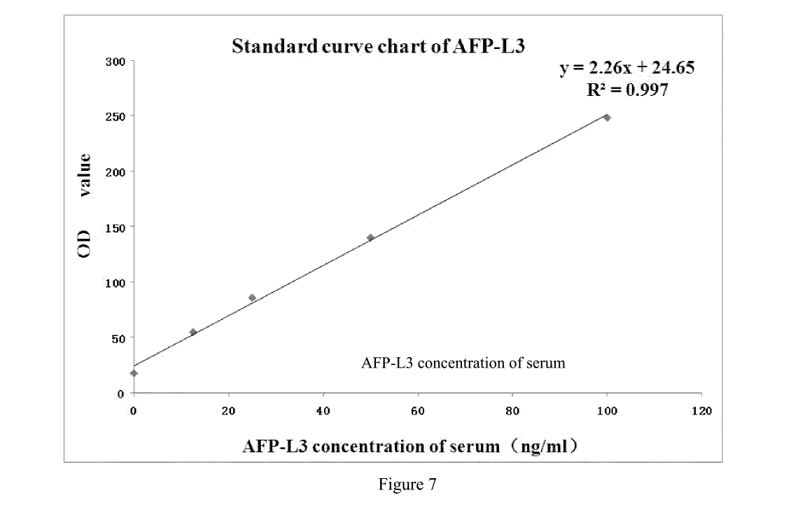

The operation procedure and protein chips in the example 1 are used to detect each concentration gradient of the serum (AFP-L3) standard substance, and the detection scan result is shown in FIG. 6. The detection results are used for drawing the standard curve chart, with the concentration of the standard substance as X-coordinate and the pixel value as Y-coordinate, on a coordinate paper. Find out the corresponding concentration through the standard curve according to the pixel value of the sample; multiply it by the dilution ratio; or calculate out the linear regression equation of the standard curve using the concentration and OD value of the standard substance, plug the OD value of the sample into the equation to calculate out the concentration of the sample, and multiple it by the dilution ratio to obtain the actual concentration of the sample. The standard curve and the regression equation b are shown in FIG. 7.

### Example 3

### Sample Detection for Verifying Stability, Accuracy and Reliability of the Method Described Herein

### Serum Samples:

39 hepatic cancer serums: from the specimen repository of Beijing Youan Hospital, Capital Medical University;

32 normal healthy human serums;

9 blank controls (blank control is 1×PBS).

Detection procedure is identical to that in example 1.

Plug the pixel value of the sample into the regression equation a in FIG. 4 to calculate out the concentration of AFP in the sample, and multiply it by the dilution ratio to obtain the total concentration of AFP in the sample. Plug the pixel value of the sample into the regression equation b in FIG. 7 to calculate out the concentration of AFP-L3 in the sample, and multiply it by the dilution ratio to obtain the total concentration of AFP-L3 in the sample.

Each chip includes 10 detection subareas, including healthy serum samples, hepatic cancer serum samples and blank controls. For details, see chip numbers and detection subarea numbers in Table 1.

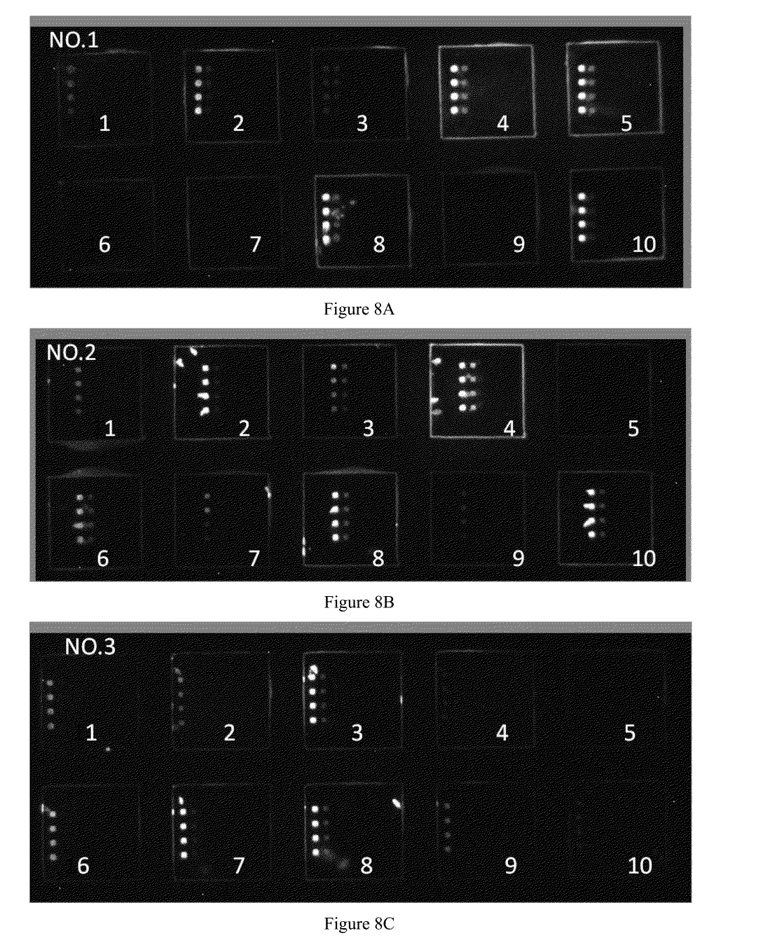

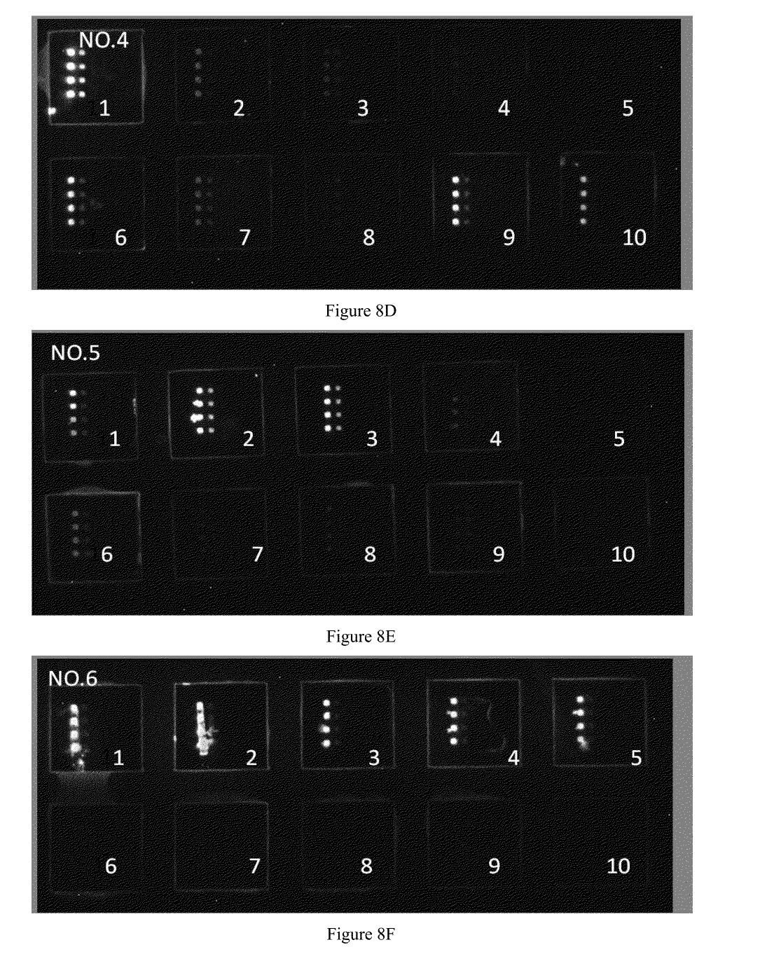

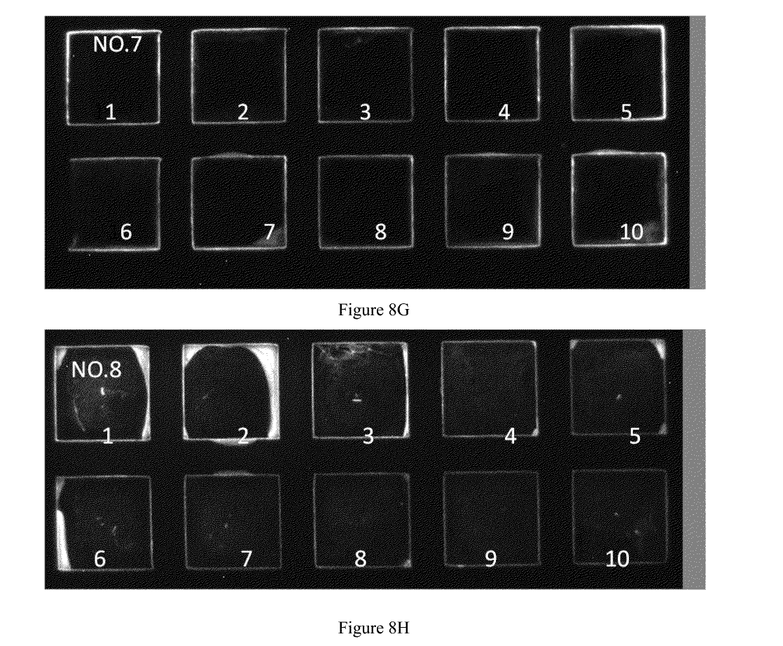

The scan chart of detection results of the samples is shown in FIG. 8.

Calculation formula:

AFP total concentration X={(scanning pixel value−Y−39.05)/5.476}}×dilution ratio;

AFF-L3 total concentration X={(scanning pixel value Y−24.65)/2.26}×dilution ratio;

AFP-L3 index=AFP-L3/AFP.

The detection result is shown in Table 1 below.

TABLE 1
Summary of detection results of clinical samples

| Chip | Grip | | AFP | AFP-L3 | AFP | AFP-L3 | |
|---|---|---|---|---|---|---|---|
| NO. | NO. | Samples | OD Value | OD Value | (ng/mL) | (ng/mL) | AFP-L3/AFP |
| No. 1 | 1 | HCC | 55 | 20 | 11.65084 | — | — |
| | 2 | HCC | 230 | 40 | 139.4814 | 27.16814 | 0.19478 |
| | 3 | HCC | 80 | 30 | 29.91234 | 9.469027 | 0.316559 |
| | 4 | HCC | 255 | 90 | 157.7429 | 115.6637 | 0.733242 |
| | 5 | HCC | 255 | 100 | 157.7429 | 133.3628 | 0.845444 |
| | 6 | C | 21 | 10 | — | — | — |
| | 7 | N | 17 | 10 | — | — | — |
| | 8 | HCC | 255 | 65 | 157.7429 | 71.41593 | 0.452736 |
| | 9 | N | 20 | 18 | — | — | — |
| | 10 | HCC | 245 | 18 | 150.4383 | — | — |
| No. 2 | 1 | HCC | 90 | 12 | 37.21695 | — | — |
| | 2 | HCC | 255 | 56 | 157.7429 | 55.48673 | 0.351754 |
| | 3 | HCC | 150 | 49 | 81.04456 | 43.09735 | 0.531773 |
| | 4 | HCC | 255 | 140 | 157.7429 | 204.1593 | 1.294254 |
| | 5 | C | 10 | 10 | — | — | — |
| | 6 | HCC | 160 | 50 | 88.34916 | 44.86726 | 0.50784 |
| | 7 | HCC | 83 | 10 | 32.10373 | — | — |
| | 8 | HCC | 255 | 70 | 157.7429 | 80.26549 | 0.508837 |
| | 9 | N | 30 | 14 | — | — | — |
| | 10 | HCC | 255 | 60 | 157.7429 | 62.56637 | 0.396635 |
| No. 3 | 1 | HCC | 130 | 10 | 66.43535 | — | — |
| | 2 | HCC | 80 | 10 | 29.91234 | — | — |
| | 3 | HCC | 250 | 63 | 154.0906 | 67.87611 | 0.440495 |
| | 4 | HCC | 25 | 10 | — | — | — |
| | 5 | C | 10 | 10 | — | — | — |
| | 6 | HCC | 160 | 10 | 88.34916 | — | — |
| | 7 | HCC | 255 | 34 | 157.7429 | 16.54867 | 0.104909 |
| | 8 | HCC | 255 | 60 | 157.7429 | 62.56637 | 0.396635 |
| | 9 | HCC | 70 | 10 | 22.60774 | — | — |
| | 10 | HCC | 50 | 10 | 7.998539 | — | — |
| No. 4 | 1 | HCC | 255 | 230 | 157.7429 | 363.4513 | 2.304074 |
| | 2 | HCC | 64 | 20 | 18.22498 | — | — |
| | 3 | N | 20 | 20 | — | — | — |
| | 4 | N | 20 | 10 | — | — | — |
| | 5 | C | 10 | 10 | — | — | — |
| | 6 | HCC | 255 | 70 | 157.7429 | 80.26549 | 0.508837 |
| | 7 | HCC | 90 | 40 | 37.21695 | 27.16814 | 0.729994 |
| | 8 | N | 20 | 20 | — | — | — |
| | 9 | HCC | 255 | 70 | 157.7429 | 80.26549 | 0.508837 |
| | 10 | HCC | 230 | 12 | 139.4814 | — | — |
| No. 5 | 1 | HCC | 255 | 55 | 157.7429 | 53.71681 | 0.340534 |
| | 2 | HCC | 255 | 197 | 157.7429 | 305.0442 | 1.933807 |
| | 3 | HCC | 255 | 180 | 157.7429 | 274.9558 | 1.743063 |
| | 4 | N | 40 | 20 | 0.693937 | — | — |
| | 5 | C | 10 | 10 | — | — | — |
| | 6 | HCC | 76 | 30 | 26.9905 | 9.469027 | 0.350828 |
| | 7 | N | 20 | 13 | — | — | — |
| | 8 | N | 30 | 10 | — | — | — |
| | 9 | N | 20 | 20 | — | — | — |
| | 10 | C | 10 | 10 | — | — | — |
| No. 6 | 1 | HCC | 255 | 30 | 157.7429 | 9.469027 | 0.060028 |
| | 2 | HCC | 255 | 27 | 157.7429 | 4.159292 | 0.026368 |
| | 3 | HCC | 255 | 27 | 157.7429 | 4.159292 | 0.026368 |
| | 4 | HCC | 255 | 30 | 157.7429 | 9.469027 | 0.060028 |
| | 5 | HCC | 255 | 20 | 157.7429 | — | — |
| | 6 | C | 10 | 10 | — | — | — |
| | 7 | N | 10 | 10 | — | — | — |
| | 8 | N | 10 | 10 | — | — | — |
| | 9 | N | 10 | 10 | — | — | — |
| | 10 | N | 10 | 10 | — | — | — |
| No. 7 | 1 | N | 10 | 10 | — | — | — |
| | 2 | N | 10 | 10 | — | — | — |
| | 3 | N | 10 | 10 | — | — | — |
| | 4 | N | 10 | 10 | — | — | — |
| | 5 | C | 10 | 10 | — | — | — |
| | 6 | N | 10 | 10 | — | — | — |
| | 7 | N | 10 | 10 | — | — | — |
| | 8 | N | 10 | 10 | — | — | — |
| | 9 | N | 10 | 10 | — | — | — |
| | 10 | N | 10 | 10 | — | — | — |
| No. 8 | 1 | N | 10 | 10 | — | — | — |
| | 2 | N | 10 | 10 | — | — | — |
| | 3 | N | 10 | 10 | — | — | — |
| | 4 | N | 10 | 10 | — | — | — |
| | 5 | C | 10 | 10 | — | — | — |
| | 6 | N | 10 | 10 | — | — | — |
| | 7 | N | 10 | 10 | — | — | — |
| | 8 | N | 10 | 10 | — | — | — |
| | 9 | N | 10 | 10 | — | — | — |
| | 10 | N | 10 | 10 | — | — | — |

HCC: HCC serum; N: normal, healthy human serum; C: blank.
—: no signal

TABLE 2
AFP-L3/AFP of 80 serum samples

| | | | | AFP-L3/ | AFP-L3/ |
|---|---|---|---|---|---|
| | AFP | AFP | | AFP | AFP |
| | ≧20 ng/ml | <20 ng/ml | AFP-L3 | ≧10% | <10% |
| HCC (39) | 35 | 2 | 26 | 22 | 4 |
| Healthy | 0 | 1 | 0 | 0 | 0 |
| (32) | | | | | |
| Blank | 0 | 0 | 0 | 0 | 0 |
| controls | | | | | |
| (9) | | | | | |

Currently, the AFP detection level adopts 20 ng/ml as the boundary, and the AFP level of normal people is lower than 20 ng/ml. AFP-L3(%)>10-15% is the positive judgment indicator.

Detection results of this chip:

No AFP or AFP-L3 is detected in 9 blank controls, which indicates the chip adopted in this experiment is effective.

No AFP or AFP-L3 is detected in 32 healthy serums, which indicates the false positive rate detected by the chip and the method disclosed herein is 0.

AFP was detected in 37 of 39 HCC samples (94.87%). AFP level greater than 20 ng/ml was found in 35 of 39 HCC samples (89.74%). Both AFP and AFP-L3 were detected in 26 of 39 HCC samples. AFP and AFP-L3 were both undetected in 2 of 39 HCC samples. AFP-L3/AFP ratio greater than 10% was found in 22 of 26 HCC samples (84.61%), while AFP-L3/AFP ratio smaller than 10% was found in 4 of 26 samples. Thus, the protein microarray assay showed a sensitivity of 89.74% and a specificity of 100% for detecting AFP. It has reliable clinical application value.

The above data demonstrates that the chip and the method described herein have favorable stability, accuracy and reliability.

The AFP-L3/AFP ratios of 4 samples in detection are greater than 1, because the AFP concentrations of the samples are too high and far more than 169 ng/ml, much higher than the upper limit of the pixel analysis of this chip, 255. In actual detection, a serum with high AFP concentration can be diluted in multiple proportions, so that the actual AFP concentration of this serum can be detected.

What is claimed is:

1. A chemiluminescent protein chip for detecting seroglycoid fucosylation index, wherein
a substrate slide of said chemiluminescent protein chip comprises at least one detection subarea, and said at least one detection subarea is used for detecting one serum sample;
said at least one detection subarea comprises two detection spot areas and one control spot area, wherein one of the detection spot areas comprises detection spots formed by fixed alpha fetoprotein (AFP)-specific antibodies, and the other detection spot area comprises detection spots formed by fixed lens culinaris lectin, and wherein the control spot area comprises control spots formed by fixed bovine serum albumin (BSA); and
wherein substances on all detection spots in the same detection spot area have the same concentration.

2. The chemiluminescent protein chip according to claim 1, wherein at least one of said detection spot areas comprises two detection spots.

3. The chemiluminescent protein chip according to claim 1, wherein said AFP-specific antibodies are mouse anti-human AFP antibodies.

4. The chemiluminescent protein chip according to claim 1, wherein said substrate slide comprises at least two detection subareas, and wherein each of the detection spot areas comprises four detection spots arranged in one row, and wherein said control spot area comprises four control spots arranged in one row; and wherein said four detection spots and said four control spots are arranged in three parallel rows.

5. The chemiluminescent protein chip according to claim 4, wherein said chemiluminescent protein chip comprises a bulge between each of the at least two detection subareas, wherein said bulge is a physical partition.

6. A chemiluminescent kit for detecting seroglycoid fucosylation index, wherein said chemiluminescent kit comprises the chemiluminescent protein chip according to claim 1.

7. The chemiluminescent kit according to claim 6, wherein said chemiluminescent kit further comprises an alpha fetoprotein (AFP) standard substance, biotin-labeled AFP polyclonal antibodies, avidin horseradish peroxidase (HRP) and an HRP chemiluminescent substrate solution.

8. The chemiluminescent kit according to claim 7, wherein said biotin-labeled alpha fetoprotein (AFP) polyclonal antibodies are rabbit antibodies.

9. The chemiluminescent kit according to claim 8, wherein said biotin-labeled AFP polyclonal antibodies are from a species different from that of the AFP-specific antibodies fixed on the detection spots.

10. The chemiluminescent kit according to claim 6, wherein said chemiluminescent kit further comprises conventional reagents used for washing and dilution.

11. The chemiluminescent kit according to claim 10, wherein said conventional reagents comprise Phosphate Buffered Saline (PBS) and PBS containing Tween® 20 (PBST).

12. A method of detecting alpha fetoprotein (AFP) and/or fucosylated AFP (FucAFP) and/or seroglycoid fucosylation index comprising using the kit according to claim 6.

13. A method for quantitatively detecting a fucosylation index comprising the following steps:
(a) diluting a serum sample to be detected, resulting in a diluted serum sample;
(b) adding said diluted serum sample on the at least one detection subarea of the chemiluminescent protein chip of claim 1;
(c) incubating said diluted serum sample;
(d) washing said at least one detection subarea with a washing reagent to remove nonspecific conjugates;
(e) adding biotin-labeled alpha fetoprotein (AFP) antibodies diluted with PBS to said at least one detection subarea;
(f) incubating said diluted serum sample and said biotin-labeled AFP antibodies;
(g) washing said at least one detection subarea with a washing reagent to remove nonspecific conjugates;
(h) adding avidin horseradish peroxidase (HRP) diluted with PBS to said at least one detection subarea;
(i) incubating said diluted serum sample, said biotin-labeled AFP antibodies, and said avidin HRP;
(j) washing said at least one detection subarea with a washing reagent to remove nonspecific conjugates;
(k) adding HRP chemiluminescent substrate solution to said at least one detection subarea;
(l) scanning said chemiluminescent protein chip with a chemiluminescent scanner to obtain a chemiluminescence pixel value of AFP and a chemiluminescence pixel value of fucosylated protein in said diluted serum sample;
(m) obtaining standard curve equations of AFP and fucosylated protein, wherein
the X-coordinate of a standard curve equation of AFP is a gradient of concentration values of the AFP standard substance;
the Y-coordinate of said standard curve equation of AFP is a series of chemiluminescence pixel values of AFP as detected in the step (l) by using the AFP standard substances with gradient concentrations as a series of samples to be detected; and
the X-coordinate of a standard curve equation of fucosylated protein is a gradient of concentration values of AFP-L3 in the AFP-L3 standard substance;
the Y-coordinate of said standard curve equation of fucosylated protein is a series of chemiluminescence pixel values of fucosylated protein as detected in step (l) by using AFP-L3 standard substances with gradient concentrations as a series of samples to be detected; wherein each of said AFP-L3 standard substance is a serum containing fucosylated alpha fetoprotein (AFP);
(n) plugging said chemiluminescence pixel value of AFP obtained in step (l) into said standard curve equation of AFP to obtain the AFP concentration of said diluted serum;
(o) multiplying said AFP concentration of said diluted serum with a dilution ratio of said serum sample to obtain the AFP concentration of the serum to be detected;
(p) plugging said chemiluminescence pixel value of fucosylated protein obtained in step (l) into said standard curve equation of fucosylated protein to obtain fucosylated protein concentration of said diluted serum;
(r) multiplying said fucosylated protein concentration of said diluted serum with said dilution ratio of said serum sample to obtain fucosylated protein concentration of the serum to be detected; wherein
the ratio of the fucosylated protein concentration of the serum to be detected to the fucosylated protein concentration of the serum to be detected is the fucosylation index.

14. The method according to claim 13, wherein the incubating step (c) takes place for 30 min at 37° C.

15. The method according to claim 13, wherein the incubating step (f) takes place for 30 min at 37° C.

16. The method according to claim 13, wherein the incubating step (i) takes place for 30 min at 37° C.
